SEQUENCE LISTING (2 sequences)

SEQ ID NO: 1 - protein, 212 aa, Homo sp.
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - protein, 183 aa, Homo sp.
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

(19) United States
(12) Patent Application Publication
(10) Pub. No.: US 2026/0174834 A1
(43) Pub. Date: Jun. 25, 2026

(54) LOW DOSE IL-6 FORMULATIONS AND METHODS OF USE THEREOF

(71) Applicant: SONNET BIOTHERAPEUTICS, INC., Princeton, NJ (US)

(72) Inventors: John K. CINI, Blairstown, NJ (US); Himanshu GADGIL, Princeton, NJ (US); Shilpa GADGIL, Princeton, NJ (US); Nildip CHAUHAN, Princeton, NJ (US); Varda KOLAPKAR, Princeton, NJ (US)

(21) Appl. No.: 19/114,181

(22) PCT Filed: Sep. 29, 2023
(86) PCT No.: PCT/US2023/075593

§ 371 Date: Mar. 21, 2025

Related U.S. Application Data
(60) Provisional application No. 63/377,971, filed on Sep. 30, 2022.

(51) Int. Cl. A61K 38/20; A61K 47/18; A61K 47/22; A61K 47/26

(52) U.S. Cl. CPC A61K 38/204; A61K 47/183; A61K 47/22; A61K 47/26

(57) ABSTRACT

The present disclosure provides an IL-6 formulation, comprising about 40 μg/mL to about 2 mg/ml IL-6, about 5 to about 15 mM histidine, about 5 to about 15 mg/mL glycine, about 40 to about 60 mg/mL trehalose, about 0.1 to about 0.3 mg/mL polysorbate 20, and about 1 to about 1000 μM DTPA, wherein the formulation has a pH of about 6.0 to about 8.0. Such formulations may be useful to treat and/or prevent a disease or disorder in a subject in need thereof.

### CROSS-REFERENCE TO RELATED APPLICATIONS

The present application is a national stage application of International Patent Application No. PCT/US23/75593, filed on Sep. 29, 2023, which claims priority to U.S. Provisional Patent Application No. 63/377,971, filed on Sep. 30, 2022, all of which are hereby incorporated by reference in their entirety.

### FIELD

The present disclosure generally provides IL-6 formulations including, stable low-dose formulations that are amenable to both frozen liquid and lyophilized storage, and method of use thereof including for the treatment of IL-6-related diseases and/or disorders.

### SEQUENCE LISTING

The Sequence Listing associated with this application is provided in XML format in lieu of a paper copy, and is hereby incorporated by reference into the specification. The name of the XML file containing the Sequence Listing is 116076-5019-US_Sequence_Listing.xml. The text, file is about 4,096 bytes, was created on or about Mar. 18, 2025, and is being submitted electronically via Patent Center.

### BACKGROUND

Interleukin-6 (hereinafter “IL-6”) (also known as interferon-β₂; B-cell differentiation factor; B-cell stimulatory factor-2; hepatocyte stimulatory factor; hybridoma growth factor; and plasmacytoma growth factor) is a member of a family of cytokines that promote cellular responses through a receptor complex consisting of at least one subunit of the signal-transducing glycoprotein gp130 and the IL-6 receptor (“IL-6R”; also known as gp80). The IL-6R may also be present in a soluble form (“sIL-6R”). IL-6 binds to IL-6R, which then dimerizes the signal-transducing receptor gp130. See Jones, S A, J. Immunology, 175:3463-3468 (2005).

Although IL-6 cannot directly bind gp130, it can bind to IL-6R to generate a high-affinity ternary complex of IL-6/IL-6R/gp130. IL-6 binds the IL-6R with low affinity, however, IL-6R does not have an intracellular signal transduction domain therefore this ligation alone does not lead to cellular activation. Similarly, cell surface expression of IL-6R does not mean the cell is responsive to IL-6 stimulation. Proteolytic cleavage leads to the release of soluble IL-6R (sIL-6R; sgp80) which can bind circulating IL-6 and increase the half-life of IL-6. For cellular activation, IL-6 first binds to either cell bound IL-6R or sIL-6R; the heterodimeric IL-6/IL-6R complex then associates with cell surface glycoprotein gp130. The resulting tripartite heterocomplex binds another IL-6/IL-6R/gp130 and signal transduction ensues (Bravo and Heath 2000, EMBO J., 19, (11), 2399-2411; Boulanger et al., 2003, Science, 300, 5628, 2101-2104), hence both cell-bound and soluble IL-6R contribute to cellular activation. IL-6 signaling through cell bound IL-6R has been termed cis signaling whilst cellular activation via soluble IL-6R has been described as trans signaling.

The therapeutic use of IL-6 is facilitated by formulations that retain stability of IL-6 under a variety of conditions. It is important that the therapeutic formulation permits storage without an unacceptable loss of activity of the active protein, minimizes the accumulation of undesirable products such as aggregates or degraded species (e.g., fragmented, oxidized, deamidated or isomerized species), accommodates appropriate concentrations of the protein, and does not contain components that are incompatible with therapeutic applications. Additionally, previous formulations of IL-6 use chemicals that are no longer advisable by regulatory authorities for human use, such as those containing sucrose or are not stable in both liquid and lyophilized forms. Thus, there exists a need in the art for a stable aqueous pharmaceutical formulation comprising an IL-6, which is suitable for therapeutic use.

### SUMMARY

The present disclosure generally provides IL-6 formulations that are uniquely stable in both liquid and lyophilized forms and/or do not contain sucrose. Such formulation may comprise about 40 μg/mL to about 2 mg/ml (e.g., 80-240 μg/mL) 240 μg/mL IL-6, about 5 to about 15 mM histidine, about 5 to about 15 mg/mL glycine, about 40 to about 60 mg/mL trehalose, about 0.1 to about 0.3 mg/mL polysorbate 20, and about 1 to about 1000 μM DTPA (e.g., 5, 10, 15, 20, 25, 30, 35, 40, 45, 50, 55, 60, 65, 70, 75, 80, 85, 90, 95 100, 125, 150, 175, 200, 225, 250, 275, 300, 325,350, 375, 400, 425, 450, 475, 500, 525, 550, 575, 600, 625, 650, 675, 700, 725, 750, 775, 800, 825, 850, 875, 900, 925, 950, 975, or 1000 μM), wherein the formulation has a pH of about 6.0 to about 8.0.

In some embodiments, the IL-6 is human IL-6.

In some embodiments, the IL-6 comprises the sequence set forth in SEQ ID NO:1 or SEQ ID NO:2.

In some embodiments, the formulation comprises 40 μg/mL IL-6, 45 μg/mL IL-6, 50 μg/mL IL-6, 55 μg/mL IL-6, 60 μg/mL IL-6, 65 μg/mL IL-6, 70 μg/mL IL-6, 75 μg/mL IL-6, 80 μg/mL IL-6, 85 μg/mL IL-6, 90 μg/mL IL-6, 95 μg/mL IL-6, 100 μg/mL IL-6, 105 μg/mL IL-6, 110 μg/mL IL-6, 115 μg/mL IL-6, 120 μg/mL IL-6, 125 μg/mL IL-6, 130 μg/mL IL-6, 135 μg/mL IL-6, 140 μg/mL IL-6, 145 μg/mL IL-6, 150 μg/mL IL-6, 155 μg/mL IL-6, 160 μg/mL IL-6, 165 μg/mL IL-6, 170 μg/mL IL-6, 175 μg/mL IL-6, 180 μg/mL IL-6, 185 μg/mL IL-6, 190 μg/mL IL-6, 195 μg/mL IL-6, 200 μg/mL IL-6, 205 μg/mL IL-6, 210 μg/mL IL-6, 215 μg/mL IL-6, 220 μg/mL IL-6, 225 μg/mL IL-6, 230 μg/mL IL-6, 235 μg/mL IL-6, or 240 μg/mL IL-6. In another embodiment, the formulation comprises about 80 μg/mL to about 2 mg/ml IL-6.

In some embodiments, the formulation comprises 40 μg/ml IL-6.

In some embodiments, the formulation comprises 80 μg/mL IL-6.

In some embodiments, the formulation comprises 160 μg/mL IL-6.

In some embodiments, the formulation comprises 240 μg/mL IL-6.

In some embodiments, the formulation comprises about 10 mM histidine.

In some embodiments, the formulation comprises about 10 mg/mL glycine.

In some embodiments, the formulation comprises about 50 mg/mL trehalose.

In some embodiments, the formulation comprises about 0.2 mg/mL polysorbate 20.

In some embodiments, the formulation comprises about 50 μM DTPA.

In some embodiments, the formulation has a pH of about 7.0.

In some embodiments, the formulation is a liquid formulation.

In some embodiments, the formulation is a lyophilized formulation.

The present disclosure also provides an IL-6 formulation, comprising 80 to 240 μg/mL human IL-6, 10 mM histidine, 10 mg/mL glycine, 50 mg/mL trehalose, 0.2 mg/mL polysorbate 20, and 50 μM DTPA, wherein the formulation has a pH of about 7.0.

In some embodiments, the formulation comprises 40 μg/mL IL-6, 45 μg/mL IL-6, 50 μg/mL IL-6, 55 μg/mL IL-6, 60 μg/mL IL-6, 65 μg/mL IL-6, 70 μg/mL IL-6, 75 μg/mL IL-6, 80 μg/mL IL-6, 85 μg/mL IL-6, 90 μg/mL IL-6, 95 μg/mL IL-6, 100 μg/mL IL-6, 105 μg/mL IL-6, 110 μg/mL IL-6, 115 μg/mL IL-6, 120 μg/mL IL-6, 125 μg/mL IL-6, 130 μg/mL IL-6, 135 μg/mL IL-6, 140 μg/mL IL-6, 145 μg/mL IL-6, 150 μg/mL IL-6, 155 μg/mL IL-6, 160 μg/mL IL-6, 165 μg/mL IL-6, 170 μg/mL IL-6, 175 μg/mL IL-6, 180 μg/mL IL-6, 185 μg/mL IL-6, 190 μg/mL IL-6, 195 μg/mL IL-6, 200 μg/mL IL-6, 205 μg/mL IL-6, 210 μg/mL IL-6, 215 μg/mL IL-6, 220 μg/mL IL-6, 225 μg/mL IL-6, 230 μg/mL IL-6, 235 μg/mL IL-6, or 240 μg/mL IL-6. In another embodiment, the formulation comprises about 40 μg/mL to about 2 mg/ml IL-6.

In some embodiments, the formulation comprises 40 μg/mL IL-6.

In some embodiments, the formulation comprises 80 μg/mL IL-6.

In some embodiments, the formulation comprises 160 μg/mL IL-6.

In some embodiments, the formulation comprises 240 μg/mL IL-6.

In some embodiments, the formulation is suitable for parenteral administration.

In some embodiments, the formulation is suitable for intravenous administration.

In some embodiments, the formulation is suitable for subcutaneous administration.

The present disclosure also provides method of treating an IL-6 related disease or disorder in a subject in need thereof by administering a formulation as disclosed herein to the subject.

### DETAILED DESCRIPTION

The present disclosure provides IL-6 (e.g., h-rIL-6) formulations including, stable low-dose formulations that are amenable to both frozen liquid and lyophilized storage, and method of use thereof including for the treatment of IL-6-related diseases and/or disorders. Advantageously, the formulated IL-6 needs is maintained in a stable presentation (e.g., without degradation for long periods of time at different temperatures, sustained multiple freezing and thawing cycles, and readily available for injection upon thawing or reconstitution with water for injection) and can be used in diabetic patients in which their diet does not allow for sugar consumption.

### 1.1 Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R.J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a formulation” includes a plurality of such formulations, so that a formulation X includes a plurality of formulations X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit.

A “stable” IL-6 formulation is one in which IL-6 substantially retains its physical stability and/or chemical stability and/or its biological activity upon storage. The storage period is generally selected based on the intended shelf-life of the formulation assessed at different temperatures (from −80° C. to +40° C.). Various analytical techniques for measuring protein stability are available in the art. Examples of the analytical techniques are described below. By “substantially retains” is intended 85% or greater retention, such as at least 90% retention or at least 95% retention depending on the analytical techniques and specifications applied.

An IL-6 protein “retains its physical stability” in a pharmaceutical formulation if it shows no significant physical changes such as aggregation, precipitation and/or denaturation upon visual examination of color and/or clarity, or as measured by UV light scattering or by size exclusion chromatography.

An IL-6 protein “retains its chemical stability” in a pharmaceutical formulation if no significant chemical changes of the protein is shown. Chemical stability can be assessed by detecting and quantifying chemically altered forms of the protein. Chemical alteration may involve size modification (e.g., clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g., occurring as a result of deamidation, oxidation and/or isomerization) which can be evaluated by ion-exchange chromatography, for example.

An IL-6 protein “retains its biological activity” in a pharmaceutical formulation if the biological activity of the protein at a given time is not significantly changed from the biological activity exhibited at the time the pharmaceutical formulation was prepared. “Biological activity” of an IL-6 protein refers to the ability of the protein to produce a measurable biological response which can be measured in vitro or in vivo.

A “histidine buffer” is a buffer comprising histidine ions. Examples of histidine buffers include histidine chloride, histidine acetate, histidine phosphate, and histidine sulfate solutions. The histidine buffer or histidine-HCl buffer has a pH of about 5.5 to about 6.5, about 5.6 to about 6.4, about 5.7 to about 6.3, about 5.8 to about 6.2, about 5.9 to about 6.1, about 6.0 to about 7.0, about 6.5 to about 7.0, about 7.0 to about 7.5, or about 7.0 to about 8.0.

By “IL-6” or “interleukin 6 (IL-6)” or “IL-6 polypeptide” is meant a polypeptide or fragment thereof having at least about 85% or greater amino acid identity to the amino acid sequence provided at NCBI Accession No. NP_000591 and having IL-6 biological activity. IL-6 is a pleotropic cytokine with multiple biologic functions. Exemplary IL-6 biological activities include immunostimulatory and pro-inflammatory activities. By “IL-6” or “interleukin 6 (IL-6) nucleic acid” is meant a polynucleotide encoding an interleukin 6 (IL-6) polypeptide. An exemplary interleukin 6 (IL-6) nucleic acid sequence is provided at NCBI Accession No. NM_000600.

Percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared. Unless otherwise specified, the percent sequence identity is determined using BLAST algorithms using default parameters.

By “subject” is meant a human or non-human mammal, including, but not limited to, bovine, equine, canine, ovine, feline, and rodent, including murine and rattus, subjects. A “patient” is a human subject.

As used herein, the terms “treat,” “treating,” “treatment,” and the like refer to reducing or ameliorating a disorder, and/or signs or symptoms associated therewith, or slowing or halting the progression thereof. It will be appreciated that, although not precluded, treating a disorder or condition does not require that the disorder, condition, or symptoms associated therewith be completely eliminated.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±1%, ±5%, or ±10%, of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than 1, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number 10”, it implies a continuous range that includes whole numbers and fractional numbers less than number 10, as discussed above.

Similarly, if the variable disclosed is a number greater than “number 10”, it implies a continuous range that includes whole numbers and fractional numbers greater than number 10.

These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

### 1.2. Formulation

In a first aspect, provided herein an aqueous or lyophilized formulation (pharmaceutical composition) comprising an IL-6 protein. In an embodiment, the formulation is sterile. In another embodiment, the formulation is stable. In some embodiments, the formulation further comprises at least one free amino acid. In some embodiments, the formulation further comprises at least one surfactant. In some embodiments, the formulation further comprises at least one buffering component. In some embodiments, the formulation further comprises at least one chelator. In some embodiments, the formulation comprises at least one saccharide. In some embodiments, the formulation comprises IL-6 (e.g., human IL-6). In certain embodiments, the formulation comprises at least one free amino acid, at least one surfactant, at least one buffering component, at least one chelator, at least one saccharide, and IL-6.

In some embodiments, the formulation comprises about 40 to about 240 μg/mL IL-6, about 5 to about 15 mM histidine, about 5 to about 15 mg/mL glycine, about 40 to about 60 mg/mL trehalose, about 0.1 to about 0.3 mg/mL polysorbate 20, and about 5 to about 100 μM DTPA, wherein the formulation has a pH of about 6.0 to about 8.0. In other embodiments, the formulation comprises 40 to about 240 μg/mL human IL-6, 10 mM histidine, 10 mg/mL glycine, 50 mg/mL trehalose, 0.2 mg/mL polysorbate 20, and 50 μM DTPA, wherein the formulation has a pH of about 7.0.

### 1.2.1. IL-6 Concentrations

In some embodiments, the IL-6 concentration in the formulation is at least about 80 μg/mL to about 2 mg/ml. In some embodiments, the IL-6 concentration in the formulation is at least about 40 μg/mL, at least about 45 μg/mL, at least about 50 μg/mL, at least about 55 μg/mL, at least about 60 μg/mL, at least about 65 μg/mL, at least about 70 μg/mL, at least about 75 μg/mL, at least about 80 μg/mL, at least about 85 μg/mL, at least about 90 μg/mL, at least about 95 μg/mL, at least about 100 μg/mL, at least about 105 μg/mL, at least about 110 μg/mL, at least about 115 μg/mL, at least about 120 μg/mL, at least about 125 μg/mL, at least about 130 μg/mL, at least about 135 μg/mL, at least about 140 μg/mL, at least about 145 μg/mL, at least about 150 μg/mL, at least about 155 μg/mL, at least about 160 μg/mL, at least about 165 μg/mL, at least about 170 μg/mL, at least about 175 μg/mL, at least about 180 μg/mL, at least about 185 μg/mL, at least about 190 μg/mL, at least about 195 μg/mL, at least about 200 μg/mL, at least about 205 μg/mL, at least about 210 μg/mL, at least about 215 μg/mL, at least about 220 μg/mL, at least about 225 μg/mL, at least about 230 μg/mL, at least about 235 μg/mL, at least about 240 μg/mL, at least about 245 μg/mL, at least about 250 μg/mL, at least about 255 μg/mL, at least about 260 μg/mL, at least about 265 μg/mL, at least about 270 μg/mL, at least about 275 μg/mL, or at least about 280 μg/mL.

In some embodiments, the IL-6 concentration in the formulation is about 40 μg/mL to about 280 μg/mL. In other embodiments, the IL-6 concentration in the formulation is about 100 to about 120 μg/mL, about 120 to about 140 μg/mL, about 140 to about 160 μg/mL, about 160 to about 180 μg/mL, about 180 to about 200 μg/mL, about 200 to about 220 μg/mL, about 220 to about 240 μg/mL, about 240 to about 260 μg/mL, or about 260 to about 280 μg/mL.

### 1.2.2. Chelators

In some embodiments, the formulation comprises a chelator.

In various embodiments, the chelator is at a concentration of about 1 μM to about 1000 μM, such as about 25 μM to about 75 μM, preferably about 50 μM. In various embodiments, the chelator is at a concentration of 1 μM to 100 μM, such as 25 μM to 75 μM, preferably 50 μM.

In certain embodiments, the chelator is a naturally occurring compound. In certain other embodiments, the chelator is a synthetic compound. In some embodiments, the chelator is diethylenetriamine pentaacetic acid (DTPA).

### 1.2.3. Surfactants

In some embodiments, the formulation comprises a surfactant.

The surfactant can lower surface tension of a liquid. In some embodiments, the surfactant is a nonionic surfactant. Examples of surfactants include polysorbate (polyoxyethylene sorbitan monolaurate, for example, polysorbate 20 and polysorbate 80); TRITON (t-Octylphenoxypolyethoxyethanol); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; sorbitan monopalmitate; the MONAQUAT series (Mona Industries, Inc., Paterson, N.J.); polyethyl glycol (PEG), polypropylene glycol (PPG), and copolymers of poloxyethylene and poloxypropylene glycol (e.g. Pluronics/Poloxamer, PF68 etc.). In some of these embodiments, the surfactant is polysorbate. In certain embodiments, the polysorbate is polysorbate 20, polysorbate 40, polysorbate 60, or polysorbate 80.

In various embodiments, the composition comprises a surfactant at a concentration of about 0.001% to about 1%, such as about 0.001% to about 0.1%, about 0.005% to about 0.2%, about 0.01% to about 0.2%, or about 0.05% to about 0.1% (w/v). In various embodiments, the composition comprises a surfactant at a concentration of 0.001% to 1%, such as 0.001% to 0.1%, 0.005% to 0.2%, 0.01% to 0.2%, or 0.05% to 0.1% (w/v).

### 1.2.4. Polysorbates

In some embodiments, the formulation comprises a polysorbate.

### 1.2.4.1. Polysorbate 80

In some embodiments, the formulation comprises polysorbate 80 (PS80).

In some embodiments, the formulation comprises at least about 0.001%, at least about 0.002%, at least about 0.003%, at least about 0.005%, at least about 0.01%, at least about 0.02%, at least about 0.05%, at least about 0.1%, at least about 0.2%, at least about 0.5%, or at least about 1% (w/v) polysorbate 80. In some embodiments, the formulation comprises about 0.001% to about 1%, about 0.002% to about 0.5%, about 0.005% to about 0.2%, or about 0.01% to about 0.1% (w/v) polysorbate 80. In some embodiments, the formulation comprises about 0.001%, about 0.002%, about 0.003%, about 0.005%, about 0.01%, about 0.02%, about 0.05%, about 0.07%, about 0.1%, about 0.2%, about 0.5%, or about 1% (w/v) polysorbate 80. In certain embodiments, the formulation comprises about 0.005% to about 0.5% (w/v) polysorbate 80. In certain embodiments, the formulation comprises about 0.03% to about 0.1% (w/v) polysorbate 80. In certain embodiments, the formulation comprises about 0.05% to about 0.1% (w/v) polysorbate 80. In particular embodiments, the formulation comprises about 0.07% (w/v) polysorbate 80.

In some embodiments, the formulation comprises at least 0.001%, at least 0.002%, at least 0.003%, at least 0.005%, at least 0.01%, at least 0.02%, at least 0.05%, at least 0.1%, at least 0.2%, at least 0.5%, or at least 1% (w/v) polysorbate 80. In some embodiments, the formulation comprises 0.001% to 1%, 0.002% to 0.5%, 0.005% to 0.2%, or 0.01% to 0.1% (w/v) polysorbate 80. In some embodiments, the formulation comprises 0.001%, 0.002%, 0.003%, 0.005%, 0.01%, 0.02%, 0.05%, 0.07%, 0.1%, 0.2%, 0.5%, or 1% (w/v) polysorbate 80. In certain embodiments, the formulation comprises 0.005% to 0.5% (w/v) polysorbate 80. In certain embodiments, the formulation comprises 0.03% to 0.1% (w/v) polysorbate 80. In certain embodiments, the formulation comprises 0.05% to 0.1% (w/v) polysorbate 80. In particular embodiments, the formulation comprises 0.07% (w/v) polysorbate 80.

### 1.2.4.2. Polysorbate 60

In some embodiments, the formulation comprises polysorbate 60 (PS60).

In some embodiments, the formulation comprises at least about 0.001%, at least about 0.002%, at least about 0.003%, at least about 0.005%, at least about 0.01%, at least about 0.02%, at least about 0.05%, at least about 0.1%, at least about 0.2%, at least about 0.5%, or at least about 1% (w/v) polysorbate 60. In some embodiments, the formulation comprises about 0.001% to about 1%, about 0.002% to about 0.5%, about 0.005% to about 0.2%, or about 0.01% to about 0.1% (w/v) polysorbate 60. In some embodiments, the formulation comprises about 0.001%, about 0.002%, about 0.003%, about 0.005%, about 0.01%, about 0.02%, about 0.05%, about 0.07%, about 0.1%, about 0.2%, about 0.5%, or about 1% (w/v) polysorbate 60. In certain embodiments, the formulation comprises about 0.03% to about 0.1% (w/v) polysorbate 60. In certain embodiments, the formulation comprises about 0.05% to about 0.1% (w/v) polysorbate 60. In particular embodiments, the formulation comprises about 0.07% (w/v) polysorbate 60.

In some embodiments, the formulation comprises at least 0.001%, at least 0.002%, at least 0.003%, at least 0.005%, at least 0.01%, at least 0.02%, at least 0.05%, at least 0.1%, at least 0.2%, at least 0.5%, or at least 1% (w/v) polysorbate 60. In some embodiments, the formulation comprises 0.001% to 1%, 0.002% to 0.5%, 0.005% to 0.2%, or 0.01% to 0.1% (w/v) polysorbate 60. In some embodiments, the formulation comprises 0.001%, 0.002%, 0.003%, 0.005%, 0.01%, 0.02%, 0.05%, 0.07%, 0.1%, 0.2%, 0.5%, or 1% (w/v) polysorbate 60. In certain embodiments, the formulation comprises 0.03% to 0.1% (w/v) polysorbate 60. In certain embodiments, the formulation comprises 0.05% to 0.1% (w/v) polysorbate 60. In particular embodiments, the formulation comprises 0.07% (w/v) polysorbate 60.

### 1.2.4.3. Polysorbate 40

In some embodiments, the formulation comprises polysorbate 40 (PS40).

In some embodiments, the formulation comprises at least about 0.001%, at least about 0.002%, at least about 0.003%, at least about 0.005%, at least about 0.01%, at least about 0.02%, at least about 0.05%, at least about 0.1%, at least about 0.2%, at least about 0.5%, or at least about 1% (w/v) polysorbate 40. In some embodiments, the formulation comprises about 0.001% to about 1%, about 0.002% to about 0.5%, about 0.005% to about 0.2%, or about 0.01% to about 0.1% (w/v) polysorbate 40. In some embodiments, the formulation comprises about 0.001%, about 0.002%, about 0.003%, about 0.005%, about 0.01%, about 0.02%, about 0.05%, about 0.07%, about 0.1%, about 0.2%, about 0.5%, or about 1% (w/v) polysorbate 40. In certain embodiments, the formulation comprises about 0.03% to about 0.1% (w/v) polysorbate 40. In certain embodiments, the formulation comprises about 0.05% to about 0.1% (w/v) polysorbate 40. In particular embodiments, the formulation comprises about 0.07% (w/v) polysorbate 40.

In some embodiments, the formulation comprises at least 0.001%, at least 0.002%, at least 0.003%, at least 0.005%, at least 0.01%, at least 0.02%, at least 0.05%, at least 0.1%, at least 0.2%, at least 0.5%, or at least 1% (w/v) polysorbate 40. In some embodiments, the formulation comprises 0.001% to 1%, 0.002% to 0.5%, 0.005% to 0.2%, or 0.01% to 0.1% (w/v) polysorbate 40. In some embodiments, the formulation comprises 0.001%, 0.002%, 0.003%, 0.005%, 0.01%, 0.02%, 0.05%, 0.07%, 0.1%, 0.2%, 0.5%, or 1% (w/v) polysorbate 40. In certain embodiments, the formulation comprises 0.03% to 0.1% (w/v) polysorbate 40. In certain embodiments, the formulation comprises 0.05% to 0.1% (w/v) polysorbate 40. In particular embodiments, the formulation comprises 0.07% (w/v) polysorbate 40.

### 1.2.4.4. Polysorbate 20

In some embodiments, the formulation comprises polysorbate 20 (PS20).

In some embodiments, the formulation comprises at least about 0.001%, at least about 0.002%, at least about 0.003%, at least about 0.005%, at least about 0.01%, at least about 0.02%, at least about 0.05%, at least about 0.1%, at least about 0.2%, at least about 0.5%, or at least about 1% (w/v) polysorbate 20. In some embodiments, the formulation comprises about 0.001% to about 1%, about 0.002% to about 0.5%, about 0.005% to about 0.2%, or about 0.01% to about 0.1% (w/v) polysorbate 20. In some embodiments, the formulation comprises about 0.001%, about 0.002%, about 0.003%, about 0.005%, about 0.01%, about 0.02%, about 0.05%, about 0.07%, about 0.1%, about 0.2%, about 0.5%, or about 1% (w/v) polysorbate 20. In certain embodiments, the formulation comprises about 0.03% to about 0.1% (w/v) polysorbate 20. In certain embodiments, the formulation comprises about 0.05% to about 0.1% (w/v) polysorbate 20. In particular embodiments, the formulation comprises about 0.07% (w/v) polysorbate 20.

In some embodiments, the formulation comprises at least 0.001%, at least 0.002%, at least 0.003%, at least 0.005%, at least 0.01%, at least 0.02%, at least 0.05%, at least 0.1%, at least 0.2%, at least 0.5%, or at least 1% (w/v) polysorbate 20. In some embodiments, the formulation comprises 0.001% to 1%, 0.002% to 0.5%, 0.005% to 0.2%, or 0.01% to 0.1% (w/v) polysorbate 20. In some embodiments, the formulation comprises 0.001%, 0.002%, 0.003%, 0.005%, 0.01%, 0.02%, 0.05%, 0.07%, 0.1%, 0.2%, 0.5%, or 1% (w/v) polysorbate 20. In certain embodiments, the formulation comprises 0.03% to 0.1% (w/v) polysorbate 20. In certain embodiments, the formulation comprises 0.05% to 0.1% (w/v) polysorbate 20. In particular embodiments, the formulation comprises 0.07% (w/v) polysorbate 20.

### 1.2.5. Saccharides

In some embodiments, the composition comprises a saccharide and derivatives thereof, including monosaccharides, disaccharides, trisaccharides, polysaccharides, sugar alcohols, reducing sugars, nonreducing sugars, and the like. Examples of saccharides include glucose, mannose, trehalose, lactose, fructose, maltose, dextran, dextrin, erythritol, glycerol, arabitol, sylitol, sorbitol, mannitol, mellibiose, melezitose, raffinose, mannotriose, stachyose, maltose, lactulose, maltulose, glucitol, maltitol, lactitol, isomaltulose, and the like. In some embodiments, the saccharide is a disaccharide. In certain embodiments, the disaccharide is trehalose. In various embodiments, the disaccharide is at a concentration from about 1 to about 100 mg/mL.

### 1.2.5.1. Trehalose

In some embodiments, the formulation comprises trehalose.

In various embodiments, the formulation comprises about 1 to about 100 mg/mL trehalose. In some embodiments, the formulation comprises at least about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, or about 75 mg/mL trehalose. In specific embodiments, the formulation comprises about 50 mg/mL trehalose.

### 1.2.6. Free Amino Acids

In some embodiments, the formulation comprises at least one free amino acid. In some embodiments, the formulation comprises only one free amino acid. In some embodiments, the formulation comprises only two free amino acids. In some embodiments, the formulation comprises only three free amino acids. The free amino acid can be in the L-form, the D-form or a mixture of these forms.

In certain embodiments, the at least one free amino acid is glycine, glutamine, asparagine, histidine, arginine, or lysine. In various embodiments, the free amino acid is at a concentration of about 1 mM to about 150 mM, such as about 2 mM, 3 mM, 4 mM, 5 mM, 6 mM, 7 mM, 8 mM, 9 mM, 10 mM, 11 mM, 12 mM, 13 mM, 15 mM, 16 mM, 17 mM, 18 mM, 19 mM, or 20 mM.

In some embodiments, the formulation comprises histidine and at least one additional free amino acid selected from glycine, glutamine, asparagine, histidine, arginine, and lysine. In certain embodiments, the formulation comprises histidine and glycine.

### 1.2.6.1. Glycine

In some embodiments, the formulation comprises glycine. In various embodiments, the formulation comprises about 5 to about 15 mg/mL glycine. In some embodiments, the formulation comprises at least about 5 mg/mL, at least about 10 mg/mL, or at least about 15 mg/mL glycine. In various embodiments, the formulation comprises about 5 mg/mL to about 10 mg/mL or about 10 mg/mL to about 20 mg/mL. In particular embodiments, the formulation comprises about 10 mg/mL glycine.

### 1.2.7. Buffering Agents

In some embodiments, the formulation comprises at least one buffering agent (buffering component). Typically, the buffering agent, when present, is used to adjust the pH of the formulation to about 4.0 to about 8.0, about 4.5 to about 7.5, about 5.0 to about 7.0, about 5.5 to about 6.5, about 5.7 to about 6.3, about 5.9 to about 6.1, or about 6.0.

In various embodiments the at least one buffering agent is selected from acetate, succinate, gluconate, histidine, citrate, phosphate, maleate, cacodylate, 2-[N-morpholino]ethanesulfonic acid (MES), bis(2-hydroxyethyl)iminotris [hydroxymethyl]methane (Bis-Tris), N-[2-acetamido]-2-iminodiacetic acid (ADA), glycylglycine and other organic acid buffers. In some of these embodiments, the buffering agent is histidine, citrate, phosphate, glycine, or acetate. In various embodiments, the buffering component is at a concentration from about 1 mM to about 200 mM, from about 1 mM to about 50 mM, or from about 5 mM to about 20 mM. In various embodiments, the buffering component is at a concentration from 1 mM to 200 mM, from 1 mM to 50 mM, or from 5 mM to 20 mM. In certain embodiments, the buffering component is at a concentration of about 5 mM, about 10 mM, about 15 mM, about 20 mM, or about 25 mM. In certain embodiments, the buffering component is at a concentration of 10 mM.

In a preferred embodiment, the buffering agent is histidine. In some embodiments, the formulation comprises histidine. In some embodiments, the formulation comprises L-histidine.

In some embodiments, the composition comprises at least about 1 mM, at least about 5 mM, at least about 10 mM, at least about 15 mM, at least about 20 mM, at least about 30 mM, at least about 50 mM, at least about 100 mM, at least about 150 mM, or at least about 200 mM histidine. In various embodiments, the composition comprises about 1 mM to about 200 mM, about 5 mM to about 150 mM, about 10 mM to about 100 mM, about 15 mM to about 50 mM, or about 20 mM to about 30 mM histidine. In certain embodiments, the composition comprises about 1 mM, about 5 mM, about 10 mM, about 15 mM, about 20 mM, about 30 mM, about 50 mM, about 100 mM, about 150 mM, or about 200 mM histidine. In particular embodiments, the formulation comprises about 10 mM to about 100 mM histidine. In specific embodiments, the formulation comprises about 10 mM histidine.

In some embodiments, the composition comprises at least 1 mM, at least 5 mM, at least 10 mM, at least 15 mM, at least 20 mM, at least 30 mM, at least 50 mM, at least 100 mM, at least 150 mM, or at least 200 mM histidine. In various embodiments, the composition comprises 1 mM to 200 mM, 5 mM to 150 mM, 10 mM to 100 mM, 15 mM to 50 mM, or 20 mM to 30 mM histidine. In certain embodiments, the composition comprises 1 mM, 5 mM, 10 mM, 15 mM, 20 mM, 30 mM, 50 mM, 100 mM, 150 mM, or 200 mM histidine. In particular embodiments, the formulation comprises 10 mM to 100 mM histidine. In specific embodiments, the formulation comprises 10 mM histidine.

1.2.7.1. pH

In some embodiments, the formulation has a pH of about 4.0 to about 8.0, such as about 4.5 to about 7.5, about 5.0 to about 7.0, about 5.5 to about 6.5, about 5.7 to about 6.3, or about 5.9 to about 6.1. In certain embodiments, the formulation has a pH of about 4.0, about 4.5, about 5.0, about 5.5, about 5.7, about 5.9, about 6.0, about 6.1, about 6.3, about 6.5, about 7.0, about 7.5, or about 8.0. In particular embodiments, the formulation has a pH of about 5.0 to about 7.0. In particular embodiments, the formulation has a pH of about 5.0 to about 7.0. In certain embodiments, the formulation has a pH of about 5.5 to about 6.5. In certain embodiments, the formulation has a pH of about 5.7 to about 6.3. In specific embodiments, the formulation has a pH of about 6.0.

In some embodiments, the formulation has a pH of 4.0 to 8.0, such as 4.5 to 7.5, 5.0 to 7.0, 5.5 to 6.5, 5.7 to 6.3, or 5.9 to 6.1. In certain embodiments, the formulation has a pH of 4.0, 4.5, 5.0, 5.5, 5.7, 5.9, 6.0, 6.1, 6.3, 6.5, 7.0, 7.5, or 8.0. In particular embodiments, the formulation has a pH of 5.0 to 7.0. In certain embodiments, the formulation has a pH of 5.5 to 6.5. In certain embodiments, the formulation has a pH of 5.7 to 6.3. In specific embodiments, the formulation has a pH of 6.0.

### 1.2.8. Preservatives

In some embodiments, the formulation further comprises at least one preservative. In various embodiments, the at least one preservative is selected from octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol.

### 1.2.9. Viscosity

In some embodiments, the viscosity of the formulation is less than 50 cP at 25° C., such as less than 40 cP, less than 30 cP, less than 20 cP, less than 10 cP, or less than 5 cP at 25° C. In specific embodiments, the formulation has a viscosity of less than 10 cP at 25° C. In various embodiments, the viscosity of the formulation is 1 cP, 2 cP, 3 cP, 4 cP, 5 cP, 10 cP, 15 cP, 20 cP, 25 cP, 30 cP, 35 cP, or 40 cP at 25° C.

### 1.2.10. Aggregation & Post Peak Percentage

In some embodiments, the formulation reduces the aggregation of the IL-6 protein. In certain embodiments, the formulation reduces the formation of soluble aggregates. In certain embodiments, the formulation reduces the formation of insoluble aggregates. In certain embodiments, the formulation reduces the formation of soluble aggregates compared to a formulation with less polysorbate, as measured by size exclusion-high-performance liquid chromatography (SEC-HPLC). In certain embodiments, the formulation reduces the formation of insoluble aggregates compared to a formulation with less polysorbate, as measured by visual appearance check. In certain embodiments, the formulation reduces the formation of soluble aggregates compared to a formulation without histidine, glycine, or DTPA, as measured by RP-HPLC. In some embodiments, the formulation has less than 15% soluble aggregates after 20 hours of agitation at 300 rpm, as measured by RP-HPLC. In some embodiments, the formulation has less than 10% soluble aggregates after 20 hours of agitation at 300 rpm, as measured by RP-HPLC. In some embodiments, the formulation has less than 5% soluble aggregates after 20 hours of agitation at 300 rpm, as measured by RP-HPLC. In some embodiments, the formulation has less than 2% soluble aggregates after 20 hours of agitation at 300 rpm, as measured by RP-HPLC.

In some embodiments, there is no significant increase in post peak % measured by reversed phase high performance liquid chromatography (RP-HPLC) after 14 days at −80° C. In certain embodiments, the post peak percentage is about 3 after 14 days at −80° C. measured by RP-HPLC. In other embodiments, there is no significant increase in post peak % measured by RP-HPLC data of 14 days at 2-8° C. In certain embodiments, the post peak percentage is about 3 after 14 days at 2-8° C. measured by RP-HPLC.

### 1.2.11. Potency

In some embodiments, the formulation preserves the potency of the IL-6 protein. In various embodiments, the potency of an IL-6 protein is measured by RP-HPLC.

In some embodiments, the formulation has less than 50% reduction in bioactivity (e.g., relative potency) after storage at 5±3° C. for 12 months, as measured by a reporter gene assay. In various embodiments, the formulation has less than 50%, less than 40%, less than 30%, less than 20%, or less than 10% reduction in bioactivity after storage at 5±3° C. for 12 months, as measured by RP-HPLC. In certain embodiments, the formulation has less than 30% reduction in bioactivity after storage at 5±3° C. for 12 months, as measured by RP-HPLC. In certain embodiments, the formulation has less than 20% reduction in bioactivity after storage at 5±3° C. for 12 months, as measured by RP-HPLC. In certain embodiments, the formulation has less than 10% reduction in bioactivity after storage at 5±3° C. for 12 months, as measured by RP-HPLC.

### 1.3. Administration of the Formulation

The suitable routes of administration for the IL-6 formulations described herein include are but not limited to, parenterally (such as by subcutaneous, intravenous, intramuscular, intradermal, or intrasternal injection or infusion (e.g., as sterile injectable aqueous or non-aqueous solutions or suspensions, etc.)). In certain embodiments, the formulations are suitable for parenteral injection. In certain embodiments, the formulations are suitable for intravenous injection. In certain embodiments, the formulations are suitable for subcutaneous injection.

In some embodiments, the formulations are suitable for administration in single or multiple doses.

### 1.4. Dosage Form

In another aspect, provided herein are dosage forms containing one or more unit doses of a pharmaceutical composition comprising IL-6.

In various embodiments, the dosage form is a pre-filled syringe. In various embodiments, the dosage form is an auto-inject pen. In some embodiments, the dosage form is a device having automatized administration.

In various embodiments, the dosage form comprises one or more unit doses of a formulation as described hereinabove. In typical embodiments, the formulation is a liquid formulation. In other embodiments, the formulation is a dry formulation, including but not limited to a lyophilate. In particular embodiments, the dosage form comprises a dry formulation and a measured quantity of aqueous diluent.

In some embodiments, the unit dosage form comprises a formulation comprising about 40 to about 240 μg/mL IL-6, about 5 to about 15 mM histidine, about 5 to about 15 mg/mL glycine, about 40 to about 60 mg/mL trehalose, about 0.1 to about 0.3 mg/mL polysorbate 20, and about 5 to about 100 μM DTPA, wherein the formulation has a pH of about 6.0 to about 8.0.

### 1.5. Methods of Treatment

In another aspect, provided herein are methods of treating a disease or disorder in a patient comprising administering to the patient the IL-6 formulation described herein. In some embodiments, the patient has a disease or disorder that can be treated or alleviated by IL-6 administration, including, for example, chemotherapy induced peripheral neuropathies (CIPN), frailty, or diabetic peripheral neuropathies (DPN).

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### EXAMPLES

### Example 1: Drug Substance Design of Experiments Study

The IL-6 formulation (drug substance “DS” formulation) has 10 mM histidine, 10 mg/mL glycine, 50 mg/mL trehalose, 0.2 mg/mL polysorbate 20 and 50 μM DTPA, pH 7.0. A design of experiment (DOE) study was done using finalized DS formulation. Two factor-two level DOE study was conducted; factor 1 with pH 7.0±0.5 range and factor 2 with protein concentration 5±3 mg/mL. The details of DOE combinations are mentioned in Table 1.

TABLE 1
DS DOE combinations

| | | | Protein | | |
|---|---|---|---|---|---|
| | | | concentration | | |
| | | pH | (mg/mL) | | |
| Formulation | Buffer | (factor 1) | (factor 2) | Stabilizers | Surfactant |
| Formulation | 10 mM | 7.5 | 8 | 10 mg/mL glycine + | 0.2 mg/mL |
| 1 (+, +) | Histidine | | | 50 mg/mL trehalose | Polysorbate 20 |
| Formulation | | 6.5 | | 10 mg/mL glycine + | 0.2 mg/mL |
| 2 (−, +) | | | | 50 mg/mL trehalose | Polysorbate 20 |
| Formulation | | 6.5 | 2 | 10 mg/mL glycine + | 0.2 mg/mL |
| 3 (−, −) | | | | 50 mg/mL trehalose | Polysorbate 20 |
| Formulation | | 7.5 | | 10 mg/mL glycine + | 0.2 mg/mL |
| 4 (+, −) | | | | 50 mg/mL trehalose | Polysorbate 20 |
| Formulation | | 7.0 | 5 | 10 mg/mL glycine + | 0.2 mg/mL |
| 5 (target) | | | | 50 mg/mL trehalose | Polysorbate 20 |
| Formulation | | 7.0 | | 10 mg/mL glycine + | NA |
| 6 (target, | | | | 50 mg/mL trehalose | |
| no poly20) | | | | | |

Six different buffers of selected DS formulation were prepared. All the buffers were prepared and compositions are captured in subsequent Tables 3 to 7. IB022 standard provided by Sonnet BioTherapeutics is having buffer components as 40 mM phosphate and excipient as 10 mM NaCl of pH 7.0. For performing DOE study, IB022 was buffer exchanged in six prepared buffers. After buffer exchange, retentate samples were analysed for protein content and pH. Protein content and pH of retentate samples are captured in Table 8 (Sample ID). Retentate was diluted to the target protein concentration based on DOE factor (as mentioned in Table 2) if required. All the six formulations were filtered and filled in 5 mL PETG bottles as 1.5 mL fill volume. These six formulations were charged for stability study for 14 days at temperatures 2-8° C. (stress) and −80° C. (real time). The plan of stage IV excipient screening is mentioned in Table 2 below.

TABLE 2
Stage IV DS DOE plan

| | | | | Protein | | | Stability |
|---|---|---|---|---|---|---|---|
| | | | | concentration | | | temperature |
| | | | pH | (mg/mL) | | | and time |
| Sample ID | Formulation | Buffer | (factor 1) | (factor 2) | Stabilizers | Surfactant | points |
| SON080F14 | Formulation | 10 mM | 7.5 | 8 | 10 | 0.2 | control |
| a | 1 (+, +) | Histidine | | | mg/mL | mg/mL | (−80° C.) |
| | | | | | glycine + | Polysorbate | and stress |
| | | | | | 50 | 20 | stability |
| | | | | | mg/mL | | (5° C.) for |
| | | | | | trehalose | | 0, 3, 7, 14 |
| SON080F14 | Formulation | 10 mM | 6.5 | 8 | 10 | 0.2 | days |
| b | 2 (+, −) | Histidine | | | mg/mL | mg/mL | |
| | | | | | glycine + | Polysorbate | |
| | | | | | 50 | 20 | |
| | | | | | mg/mL | | |
| | | | | | trehalose | | |
| SON080F14 | Formulation | 10 mM | 6.5 | 2 | 10 | 0.2 | |
| c | 3 (−, −) | Histidine | | | mg/mL | mg/mL | |
| | | | | | glycine + | Polysorbate | |
| | | | | | 50 | 20 | |
| | | | | | mg/mL | | |
| | | | | | trehalose | | |
| SON080F14 | Formulation | 10 mM | 7.5 | 2 | 10 | 0.2 | |
| d | 4 (−, +) | Histidine | | | mg/mL | mg/mL | |
| | | | | | glycine + | Polysorbate | |
| | | | | | 50 | 20 | |
| | | | | | mg/mL | | |
| | | | | | trehalose | | |
| SON080F14 | Formulation | 10 mM | 7.0 | 5 | 10 | 0.2 | |
| e | 5 | Histidine | | | mg/mL | mg/mL | |
| | (target) | | | | glycine + | Polysorbate | |
| | | | | | 50 | 20 | |
| | | | | | mg/mL | | |
| | | | | | trehalose | | |
| SON080F14 | Formulation | 10 mM | 7.0 | 5 | 10 | NA | |
| f | 6 | Histidine | | | mg/mL | | |
| | (target, no | | | | glycine + | | |
| | poly20) | | | | 50 | | |
| | | | | | mg/mL | | |
| | | | | | trehalose | | |

TABLE 1
Composition of SON080F14a and SON080F14d

| Excipients | mg/mL |
|---|---|
| Histidine | 1.53 |
| Histidine monohydrochloride monohydrate | 0.04 |
| Glycine | 10 |
| Trehalose | 50 |
| Poly20 | 0.2 |

pH 7.5

TABLE 4
Composition of SON080F14b and SON080F14c

| Excipients | mg/mL |
|---|---|
| Histidine | 1.1 |
| Histidine monohydrochloride monohydrate | 0.61 |
| Glycine | 10 |
| Trehalose | 50 |
| Poly20 | 0.2 |

pH 6.5

TABLE 2
Composition of SON080F14e

| Excipients | mg/mL |
|---|---|
| Histidine | 1.430 |
| Histidine monohydrochloride monohydrate | 0.17 |
| Glycine | 10 |
| Trehalose | 50 |
| Poly20 | 0.2 |

pH 7.0

TABLE 3
Composition of SON080F14f

| Excipients | mg/mL |
|---|---|
| Histidine | 1.430 |
| Histidine monohydrochloride monohydrate | 0.17 |
| Glycine | 10 |
| Trehalose | 50 |

pH 7.0

TABLE 4
Composition of reference buffer (40
mM phosphate + 10 mM NaCl, pH 7.0)

| Excipients | mg/mL |
|---|---|
| sodium phosphate monobasic monohydrate | 2.21 |
| sodium phosphate dibasic dihydrate | 4.27 |
| NaCl | 0.584 |

pH 7.0

TABLE 5
Retentate samples pH and protein content results

| | | | Protein content |
|---|---|---|---|
| | | | observed in |
| | | | retentate |
| Sample ID | Sample description | pH | (mg/mL) |
| SON080F14 | 10 mM histidine buffer + 10 mg/mL glycine + 50 | 7.5 | 7.9 |
| a retentate | mg/mL trehalose + 0.2 mg/mL Poly 20, pH 7.5 | | |
| SON080F14 | 10 mM histidine buffer + 10 mg/mL glycine + 50 | 6.5 | 10.2 |
| b retentate | mg/mL trehalose + 0.2 mg/mL Poly 20, pH 6.5 | | |
| SON080F14 | 10 mM histidine buffer + 10 mg/mL glycine + 50 | 6.5 | 3.5 |
| c retentate | mg/mL trehalose + 0.2 mg/mL Poly 20, pH 6.5 | | |
| SON080F14 | 10 mM histidine buffer + 10 mg/mL glycine + 50 | 7.5 | 3.7 |
| d retentate | mg/mL trehalose + 0.2 mg/mL Poly 20, pH 7.5 | | |
| SON080F14e | 10 mM histidine buffer + 10 mg/mL glycine + 50 | 7.0 | 8.5 |
| retentate | mg/mL trehalose + 0.2 mg/mL Poly 20, pH 7.0 | | |
| SON080F14f | 10 mM histidine buffer + 10 mg/mL glycine + 50 | 7.0 | 8.3 |
| retentate | mg/mL trehalose, pH 7.0 | | |

Stage IV DS DOE samples were processed and diluted to their target DOE protein concentrations through respective pH buffers. Filtration and DS filling in 5 mL PETG was done followed by charging at stress (2-8° C.) and real time (−80° C.) conditions for 14 days. These samples were analyzed for protein concentration, pH, SEC-HPLC, RP-HPLC and osmolality.

Purity by SEC-HPLC of DOE samples charged on stability for 14 days at temperatures −80° C. and 2-8° C. were checked and are captured in Table 9 and Table 10, respectively. Both −80° C. and 2-8° C. data for all the formulations were consistent up to 7 days. 14 days analysis was done, results are not reported as trend for purity by SEC-HPLC were not as per expectations or out of trend.

TABLE 9
Stage IV SEC-HPLC data of 14 days at −80° C.

| | | % HMW | | | | |
|---|---|---|---|---|---|---|
| | Time | % Total | | % Pre- | % Main | % |
| Sample ID | points | HMW | % HMW | peak | peak | LMW |
| IB022 | | 25.4 | 8.6 | 16.8 | 74.6 | 0 |
| SON080F14 | 0 | 22.1 | 3.5 | 18.6 | 77.9 | 0 |
| a | 7 | 21.4 | 2.9 | 18.6 | 78.6 | 0 |
| | 14 | Results not reported | | | | |
| SON080F14 | 0 | 22.4 | 4.1 | 18.3 | 77.7 | 0 |
| b | 7 | 22.0 | 3.5 | 18.5 | 78.0 | 0 |
| | 14 | Results not reported | | | | |
| SON080F14 | 0 | 21.4 | 3.2 | 18.2 | 78.6 | 0 |
| c | 7 | 21.1 | 2.7 | 18.4 | 78.9 | 0 |
| | 14 | Results not reported | | | | |
| SON080F14 | 0 | 21.7 | 3.3 | 18.4 | 78.3 | 0 |
| d | 7 | 21.5 | 2.8 | 18.7 | 78.5 | 0 |
| | 14 | Results not reported | | | | |
| SON080F14 | 0 | 20.8 | 3.5 | 17.3 | 78.3 | 0 |
| e | 7 | 21.4 | 2.8 | 18.6 | 78.6 | 0 |
| | 14 | Results not reported | | | | |
| SON080F14 | 0 | 22.0 | 3.3 | 18.9 | 78.0 | 0 |
| f | 7 | 21.4 | 2.7 | 18.6 | 78.6 | 0 |

| | |
|---|---|
| 14 | Results not reported |

TABLE 10
Stage IV SEC-HPLC data of 14 days at 2-8° C.

| | | % HMW | | | | |
|---|---|---|---|---|---|---|
| | Time | % Total | | % Pre- | % Main | % |
| Sample ID | points | HMW | % HMW | peak | peak | LMW |
| IB022 | | 25.4 | 8.6 | 16.8 | 74.6 | 0 |
| SON080F14 a | 0 | 22.1 | 3.5 | 18.6 | 77.9 | 0 |
| | 3 | 22.3 | 3.8 | 18.5 | 77.7 | 0 |
| | 7 | 21.8 | 3.3 | 18.4 | 78.2 | 0 |
| | 14 | Results not reported | | | | |
| SON080F14 b | 0 | 22.4 | 4.1 | 18.3 | 77.7 | 0 |
| | 3 | 21.2 | 2.8 | 18.4 | 78.8 | 0 |
| | 7 | 23.8 | 5.2 | 18.5 | 76.3 | 0 |
| | 14 | Results not reported | | | | |
| SON080F14 c | 0 | 21.4 | 3.2 | 18.2 | 78.6 | 0 |
| | 3 | 20.9 | 2.5 | 18.4 | 79.1 | 0 |
| | 7 | 21.4 | 3.2 | 18.2 | 78.6 | 0 |
| | 14 | Results not reported | | | | |
| SON080F14 d | 0 | 21.7 | 3.3 | 18.4 | 78.3 | 0 |
| | 3 | 21.1 | 2.9 | 18.2 | 78.9 | 0 |
| | 7 | 21.1 | 3.0 | 18.1 | 78.9 | 0 |
| | 14 | Results not reported | | | | |
| SON080F14 e | 0 | 20.8 | 3.5 | 17.3 | 78.3 | 0 |
| | 3 | 21.3 | 2.3 | 19.0 | 78.7 | 0 |
| | 7 | 21.6 | 3.1 | 18.4 | 78.4 | 0 |
| | 14 | Results not reported | | | | |
| SON080F14 f | 0 | 22.0 | 3.3 | 18.7 | 78.0 | 0 |
| | 3 | 20.9 | 1.8 | 19.0 | 79.2 | 0 |
| | 7 | 21.7 | 3.1 | 18.6 | 78.3 | 0 |

| | |
|---|---|
| 14 | Results not reported |

Purity by RP-HPLC of excipient screening samples charged on stability for 14 days at temperatures −80° C. and 2-8° C. were checked and are captured in Table 11 and Table 12, respectively. −80° C. data for all the formulations were consistent for 7 days when compared with I1B022, only in formulation SON080F14d was there an increase in % post peak at 7 days storage.

2-8° C. storage was consistent till 7 days in SON080F14a, but in other samples there was an increase in % post peak and a decrease in % main peak observed when compared with I1B022. 14 days results are not reported as trend for purity by SEC-HPLC were not as per expectations or out of trend.

TABLE 11
Stage IV RP HPLC data of 14 days at −80° C.

| | | Time | % Pre- | % Main | % Post |
|---|---|---|---|---|---|
| | Sample ID | points | peak | peak | peak |
| IB022 | | | 8.1 | 89.1 | 2.8 |
| | SON080F14 a | 0 | 8.5 | 88.6 | 3.0 |
| | | 7 | 8.6 | 88.6 | 2.8 |
| | | 14 | Results not reported | | |
| | SON080F14 b | 0 | 8.8 | 88.3 | 3.0 |
| | | 7 | 7.7 | 89.5 | 2.8 |
| | | 14 | Results not reported | | |
| | SON080F14 c | 0 | 8.6 | 88.5 | 2.9 |
| | | 7 | 7.6 | 89.5 | 2.9 |
| | | 14 | Results not reported | | |
| | SON080F14 d | 0 | 8.6 | 88.3 | 3.1 |
| | | 7 | 7.6 | 87.4 | 5.1 |
| | | 14 | Results not reported | | |
| | SON080F14 e | 0 | 8.6 | 88.4 | 3.0 |
| | | 7 | 7.4 | 89.7 | 2.9 |
| | | 14 | Results not reported | | |
| | SON080F14 f | 0 | 8.5 | 88.5 | 2.9 |
| | | 7 | 7.6 | 89.7 | 2.8 |

| | |
|---|---|
| 14 | Results not reported |

TABLE 12
Stage IV RP HPLC data of 14 days at 2-8° C.

| | | Time | % Pre- | % Main | % Post |
|---|---|---|---|---|---|
| | Sample ID | points | peak | peak | peak |
| IB022 | | | 8.1 | 89.1 | 2.8 |
| | SON080F14 a | 0 | 8.5 | 88.6 | 3.0 |
| | | 3 | 8.9 | 88.1 | 3.0 |
| | | 7 | 7.9 | 89.3 | 2.9 |
| | | 14 | Results not reported | | |
| | SON080F14 b | 0 | 8.8 | 88.3 | 3.0 |
| | | 3 | 8.6 | 88.3 | 3.1 |
| | | 7 | 7.3 | 81.6 | 11.1 |
| | | 14 | Results not reported | | |
| | SON080F14 c | 0 | 8.6 | 88.5 | 2.9 |
| | | 3 | 8.5 | 88.3 | 3.2 |
| | | 7 | 7.0 | 79.3 | 13.7 |
| | | 14 | Results not reported | | |
| | SON080F14 d | 0 | 8.6 | 88.3 | 3.1 |
| | | 3 | 8.4 | 88.4 | 3.3 |
| | | 7 | 7.3 | 82.1 | 10.7 |
| | | 14 | Results not reported | | |
| | SON080F14 e | 0 | 8.6 | 88.4 | 3.0 |
| | | 3 | 8.1 | 88.5 | 3.4 |
| | | 7 | 7.1 | 72.5 | 20.4 |
| | | 14 | Results not reported | | |
| | SON080F14 f | 0 | 8.5 | 88.5 | 2.9 |
| | | 3 | 8.6 | 88.4 | 3.0 |
| | | 7 | 8.0 | 84.7 | 7.4 |

| | |
|---|---|
| 14 | Results not reported |

DOE study was performed to study the different pH and concentration. No change in quality attributes was observed in the pH range of 6.5 to 7.5 with target pH 7.0 in concentration ranging from 2 mg/mL to 8 mg/mL based on the 7 days data. Results of 14 days are not as per expectation or out of trend, hence not considered for this report.

### Example 2: Drug Product (DP) Stability Study

Three drug product presentations 40, 120 and 240 μg/mL of liquid and lyophilized IL-6 were manufactured and charged for stress and real time stability for 14 days. Results were captured for all the three OP strengths of liquid and lyophilized OP stability up to 14 days. 120 μg/mL liquid OP data was discontinued from 3 days due to some unexpected results and hence not reported here.

Protein concentration was measured by RP-UPLC method for 40, 120 and 240 μg/mL, on OP samples charged on stability for 14 days at temperatures of 25° C. (Stress, ST) and −20° C. (Real Time, RT) for liquid formulation and 40° C. (ST) and 5° C. (RT) for lyophilized formulation. Results are captured in Table 13, Table 14 and Table 15 respectively.

Protein concentration for 40 μg/mL for both formulations up to 14 days was consistent and was around 40 μg/mL at both RT and ST conditions. Protein concentration for 120 μg/mL for both formulations up to 14 days was consistent and was around 120 μg/mL at both RT and ST conditions. ST for 120 μg/mL liquid OP data was not reported. Protein concentration for 240 μg/mL for liquid formulation up to 14 days was consistent compared to day 0 at both RT and ST, whereas for lyophilized formulation up to 14 days protein concentration was consistent compared to day 0 at both RT and ST.

TABLE 13
Protein concentration (μg/mL) data of 40 μg/mL DP up to 14 days

| | RT | | | ST | | | |
|---|---|---|---|---|---|---|---|
| Sample ID and details | 0 day | 7 day | 14 day | 0 day | 3 day | 7 day | 14 day |
| SON080F19 40 μg/mL, | 40 | 40 | 40 | 40 | 40 | 40 | 40 |
| Liquid DP | | | | | | | |
| SON080F23a 40 μg/mL, | 40 | 41 | 40 | 40 | 42 | 39 | 42 |
| Lyophilized DP | | | | | | | |

TABLE 14
Protein concentration (μg/mL) data of 120 μg/mL DP up to 14 days

| | RT | | | | ST | | | |
|---|---|---|---|---|---|---|---|---|
| Sample ID and details | 0 day | 3 day | 7 day | 14 day | 0 day | 3 day | 7 day | 14 day |
| SON080F24 120 μg/mL, | 120 | 130 | 120 | 127 | Data not reported | | | |
| Liquid DP | | | | | | | | |
| SON080F23b 120 μg/mL, | 120 | 118 | 122 | 125 | 120 | 123 | 121 | 124 |
| Lyophilized DP | | | | | | | | |

TABLE 15
Protein concentration (μg/mL) data of 240 μg/mL DP up to 14 days

| | RT | | | ST | | | |
|---|---|---|---|---|---|---|---|
| Sample ID and details | 0 day | 7 day | 14 day | 0 day | 3 day | 7 day | 14 day |
| SON080F26 240 μg/mL, | 260.7 | 262.2 | 262.4 | 260.7 | 262.2 | 259.9 | 260.1 |
| Liquid DP | | | | | | | |
| SON080F25 240 μg/mL, | 252.8 | 252.1 | 254.0 | 252.8 | 251.9 | 251.3 | 250.2 |
| Lyophilized DP | | | | | | | |

pH for 40, 120 and 240 μg/mL OP samples charged on stability for 14 days at temperatures 25° C. (ST) and −20° C. (RT) for liquid formulation and 40° C. (ST) and 5° C. (RT) for lyophilized formulation are captured in Table 16, Table 17 and Table 18 respectively. The target pH of formulation initially was 7.0 and hence OP stability of presentations 40 and 120 μg/mL were performed at pH 7.0. For 240 μg/mL, pH was updated to 7.2 and hence formulation of pH 7.2 was prepared.

pH for 40 μg/mL and 120 μg/mL for both the conditions (RT and ST) and both formulations (liquid and lyophilized) up to 14 days was consistent and was around the range of 6.8-7.3. pH for 240 μg/mL for liquid and lyophilized formulations up to 14 days was consistent around pH 7.0-7.1 at both RT and ST conditions.

TABLE 16
pH data of 40 μg/mL DP up to 14 days

| | RT | | | ST | | | |
|---|---|---|---|---|---|---|---|
| Sample ID and details | 0 day | 7 day | 14 day | 0 day | 3 day | 7 day | 14 day |
| SON080F19 40 μg/mL, | 7.0 | 6.8 | 7.0 | 7.0 | 7.0 | 6.9 | 7.0 |
| Liquid DP | | | | | | | |
| SON080F23a 40 μg/mL, | 7.3 | 6.8 | 6.9 | 7.3 | 6.9 | 6.9 | 6.8 |
| Lyophilized DP | | | | | | | |

TABLE 17
pH data of 120 μg/mL DP up to 14 days

| | RT | | | | ST | | | |
|---|---|---|---|---|---|---|---|---|
| Sample ID and details | 0 day | 3 day | 7 day | 14 day | 0 day | 3 day | 7 day | 14 day |
| SON080F24 120 μg/mL, | 6.9 | 6.8 | 6.9 | 6.9 | 6.9 | 6.8 | 6.9 | — |
| Liquid DP | | | | | | | | |
| SON080F23b 120 μg/mL, | 7.2 | 6.9 | 6.9 | 6.8 | 7.2 | 7.0 | 6.9 | 6.8 |
| Lyophilized DP | | | | | | | | |

TABLE 18
Protein concentration (μg/mL) data of 240 μg/mL DP up to 14 days

| | RT | | | ST | | | |
|---|---|---|---|---|---|---|---|
| Sample ID and details | 0 day | 7 day | 14 day | 0 day | 3 day | 7 day | 14 day |
| SON080F26 240 μg/mL, | 7.0 | 7.1 | 7.1 | 7.0 | 7.0 | 7.0 | 7.0 |
| Liquid DP | | | | | | | |
| SON080F25 240 μg/mL, | 7.1 | 7.1 | 7.0 | 7.1 | 7.1 | 7.1 | 7.1 |
| Lyophilized DP | | | | | | | |

Purity by SEC-HPLC was measured OP samples at 40, 120 and 240 μg/mL when charged on stability for 14 days at temperatures 25° C. (ST) and −20° C. (RT) for liquid formulation and 40° C. (ST) and 5° C. (RT) for lyophilized formulation. Results are captured in Table 19, Table 20 and Table 21, respectively.

Purity for 40 μg/mL for both conditions (RT and ST) and both formulations (liquid and lyophilized) up to 14 days was consistent, except in stress condition of lyophilized formulation there was increase in % HMW observed at 14 days. Purity for 120 μg/mL for liquid formulation at RT condition up to 14 days was consistent, whereas ST data was not reported. For lyophilized formulation of 120 μg/mL up to 7 days, purity data was consistent, but on day 14 both RT and ST data shows increase in % HMW. Purity for 240 μg/mL for both conditions (RT and ST) and both formulations (liquid and lyophilized) up to 14 days was consistent with no changes observed.

TABLE 19
SEC-HPLC data of 40 μg/mL DP up to 14 days

| | | | % HMW | | | | |
|---|---|---|---|---|---|---|---|
| | | Time | % Total | % | % Pre- | % Main | % |
| Sample ID | Condition | points | HMW | HMW | peak | peak | LMW |
| IB022 | NA | NA | 28.9 | 11.7 | 17.2 | 71.1 | 0 |
| SON080F19 | RT | 0 | 24.6 | 7.9 | 16.7 | 75.5 | 0 |
| 40 μg/mL, | (−20° C.) | 7 | 22.4 | 6.8 | 17.6 | 75.6 | 0 |
| Liquid DP | | 14 | 23.8 | 6.2 | 17.6 | 76.2 | 0 |
| | ST | 0 | 24.6 | 7.9 | 16.7 | 75.5 | 0 |
| | (25° C.) | 3 | 24.0 | 6.5 | 17.5 | 76.0 | 0 |
| | | 7 | 22.4 | 4.5 | 17.9 | 77.6 | 0 |
| | | 14 | 22.1 | 4.2 | 18.0 | 77.9 | 0 |
| SON080F23a | RT | 0 | 23.9 | 7.8 | 16.0 | 76.2 | 0 |
| 40 μg/mL, | (5° C.) | 3 | 25.5 | 8.5 | 17.0 | 74.5 | 0 |
| Lyophilized DP | | 7 | 24.0 | 7.6 | 16.4 | 76.0 | 0 |
| | | 14 | 25.1 | 8.5 | 16.6 | 74.9 | 0 |
| | ST | 0 | 23.9 | 7.8 | 16.0 | 76.2 | 0 |
| | (40° C.) | 3 | 24.8 | 7.8 | 17.0 | 75.2 | 0 |
| | | 7 | 25.3 | 8.8 | 16.5 | 74.7 | 0 |
| | | 14 | 29.1 | 12.4 | 16.7 | 70.9 | 0 |

TABLE 20
SEC-HPLC data of 120 μg/mL DP up to 14 days

| | | | % HMW | | | | |
|---|---|---|---|---|---|---|---|
| | | Time | % Total | % | % Pre- | % Main | % |
| Sample ID | Condition | points | HMW | HMW | peak | peak | LMW |
| IB022 | NA | NA | 28.9 | 11.7 | 17.2 | 71.1 | 0 |
| SON080F24 | RT | 0 | 24.5 | 8.3 | 16.1 | 75.6 | 0 |
| 120 μg/mL | (−20° C.) | 3 | 24.9 | 8.2 | 16.7 | 75.1 | 0 |
| Liquid DP | | 7 | 24.8 | 8.3 | 16.4 | 75.3 | 0 |
| | | 14 | 24.9 | 8.3 | 16.5 | 75.1 | 0 |
| | ST | 0 | Not reported | | | | |
| | (25° C.) | 3 | | | | | |
| | | 7 | | | | | |
| | | 14 | | | | | |
| SON080F23b | RT | 0 | 23.2 | 6.8 | 16.4 | 76.9 | 0 |
| 120 μg/mL | (5° C.) | 3 | 23.2 | 6.9 | 16.6 | 76.5 | 0 |
| Lyophilized DP | | 7 | 22.8 | 6.3 | 16.5 | 77.3 | 0 |
| | | 14 | 24.2 | 7.8 | 16.4 | 75.8 | 0 |
| | ST | 0 | 23.2 | 6.8 | 16.4 | 76.9 | 0 |
| | (40° C.) | 3 | 22.8 | 6.3 | 16.5 | 77.2 | 0 |
| | | 7 | 23.3 | 6.9 | 16.3 | 76.8 | 0 |
| | | 14 | 25.2 | 8.9 | 16.3 | 74.8 | 0 |

TABLE 21
SEC-HPLC data of 240 μg/mL DP up to 14 days

| | | | % HMW | | | | |
|---|---|---|---|---|---|---|---|
| | | Time | % Total | % | % Pre- | % Main | % |
| Sample ID | Condition | points | HMW | HMW | peak | peak | LMW |
| IB018 | NA | NA | 25.0 | 9.5 | 15.5 | 75.0 | 0 |
| SON080F26 | RT | 0 | 26.6 | 11.9 | 14.6 | 73.4 | 0 |
| 240 μg/mL, | (−20° C.) | 7 | 26.3 | 12.1 | 14.3 | 73.7 | 0 |
| Liquid DP | | 14 | 26.5 | 12.1 | 14.4 | 73.5 | 0 |
| | ST | 0 | 26.6 | 11.9 | 14.6 | 73.4 | 0 |
| | (25° C.) | 3 | 23.1 | 7.7 | 15.4 | 75.1 | 0 |
| | | 7 | 21.3 | 6.3 | 15.0 | 76.5 | 0 |
| | | 14 | 19.1 | 4.4 | 14.7 | 77.9 | 0 |
| SON080F25 | RT | 0 | 26.2 | 12.0 | 14.2 | 73.8 | 0 |
| 240 μg/mL, | (5° C.) | 7 | 26.2 | 11.8 | 14.4 | 73.8 | 0 |
| Lyophilized DP | | 14 | 26.7 | 12.1 | 14.6 | 73.3 | 0 |
| | ST | 0 | 26.2 | 12.0 | 14.2 | 73.8 | 0 |
| | (40° C.) | 3 | 26.7 | 12.3 | 14.5 | 73.3 | 0 |
| | | 7 | 26.9 | 12.4 | 14.5 | 73.1 | 0 |
| | | 14 | 26.7 | 12.5 | 14.2 | 73.3 | 0 |

Purity by RP-HPLC was measured on OP samples of 40, 120 and 240 μg/mL when charged on stability for 14 days at temperatures 25° C. (ST) and −20° C. (RT) for liquid formulation and 40° C. (ST) and 5° C. (RT) for lyophilized formulation. Results are captured in Table 22, Table 23 and Table 2824 respectively.

Purity for 40 μg/mL for both conditions (RT and ST) for lyophilized formulation up to 14 days was consistent. In contrast, there was increase in % pre-peak and % post peak observed after day 0 liquid formulation for both conditions (RT and ST) at 40 μg/mL. Purity for 120 μg/mL for liquid formulation at RT condition up to 3 days was consistent, at days 7 and 14% pre-peak was found to decrease and % main peak was increased. ST data was not reported for 120 μg/mL liquid formulation. For lyophilized formulation of 120 μg/mL at RT condition, a decrease in % pre-peak was observed at days 7 and 14, whereas for ST condition the data was consistent up to 14 days. Purity for 240 μg/mL for both conditions (RT and ST) and both formulations (liquid and lyophilized) up to 14 days was consistent with no changes observed.

TABLE 22
RP HPLC data of 40 μg/mL DP up to 14 days

| | | Time | % Pre- | % Main | % Post |
|---|---|---|---|---|---|
| Sample ID | Condition | points | peak | peak | peak |
| IB022 | NA | NA | 8.2 | 88.6 | 3.2 |
| SON080F19 | RT | 0 | 9.6 | 87.7 | 2.7 |
| 40 μg/mL, | (−20° C.) | 7 | 8.7 | 89.0 | 2.3 |
| Liquid DP | | 14 | 8.8 | 89.0 | 2.2 |
| | ST | 0 | 9.6 | 87.7 | 2.7 |
| | (25° C.) | 3 | 7.9 | 90.4 | 1.7 |
| | | 7 | 8.5 | 90.0 | 1.5 |
| | | 14 | 8.5 | 89.7 | 1.8 |
| SON080F23a | RT | 0 | 9.0 | 88.5 | 2.6 |
| 40 μg/mL, | (5° C.) | 3 | 8.5 | 88.7 | 2.8 |
| Lyophilized DP | | 7 | 8.0 | 89.2 | 2.8 |
| | | 14 | 7.6 | 89.8 | 2.6 |
| | ST | 0 | 9.0 | 88.5 | 2.6 |
| | (40° C.) | 3 | 8.7 | 88.6 | 2.7 |
| | | 7 | 8.8 | 88.7 | 2.5 |
| | | 14 | 8.6 | 89.0 | 2.5 |

TABLE 23
RP HPLC data of 120 ug/mL DP up to 14 days

| | | Time | % Pre- | % Main | % Post |
|---|---|---|---|---|---|
| Sample ID | Condition | points | peak | peak | peak |
| IB022 | NA | NA | 8.2 | 88.6 | 3.2 |
| SON080F24 | RT | 0 | 8.0 | 89.1 | 2.9 |
| 120 μg/mL, | (−20° C.) | 3 | 8.3 | 88.8 | 2.9 |
| Liquid DP | | 7 | 7.5 | 89.7 | 2.9 |
| | | 14 | 7.4 | 89.9 | 2.8 |
| | ST | 0 | Not reported | | |
| | (25° C.) | 3 | | | |
| | | 7 | | | |
| | | 14 | | | |
| SON080F23b | RT | 0 | 8.5 | 88.7 | 2.9 |
| 120 μg/mL, | (5° C.) | 3 | 8.4 | 88.5 | 3.1 |
| Lyophilized DP | | 7 | 7.8 | 89.2 | 3.0 |
| | | 14 | 7.8 | 89.2 | 3.0 |
| | ST | 0 | 8.5 | 88.7 | 2.9 |
| | (40° C.) | 3 | 8.0 | 87.3 | 4.6 |
| | | 7 | 8.1 | 88.9 | 3.0 |
| | | 14 | 8.6 | 88.7 | 2.7 |

TABLE 24
RP HPLC data of 240 μg/mL DP up to 14 days

| | | Time | % Pre- | % Main | % Post |
|---|---|---|---|---|---|
| Sample ID | Condition | points | peak | peak | peak |
| IB018 | NA | NA | 0.7 | 99.2 | 0.0 (0.040) |
| SON080F26 | RT | 0 | 0.6 | 99.3 | 0.1 |
| 240 μg/mL, | (−20° C.) | 7 | 0.6 | 99.3 | 0.1 |
| Liquid DP | | 14 | 0.5 | 99.4 | 0.1 |
| | ST | 0 | 0.6 | 99.3 | 0.1 |
| | (25° C.) | 3 | 0.6 | 99.3 | 0.1 |
| | | 7 | 0.6 | 99.3 | 0.1 |
| | | 14 | 0.6 | 99.4 | 0.1 |
| SON080F25 | RT | 0 | 0.6 | 99.3 | 0.1 |
| 240 μg/mL, | (5° C.) | 7 | 0.6 | 99.3 | 0.1 |
| Lyophilized DP | | 14 | 0.6 | 99.3 | 0.1 |
| | ST | 0 | 0.6 | 99.3 | 0.1 |
| | (40° C.) | 3 | 0.6 | 99.3 | 0.1 |
| | | 7 | 0.6 | 99.3 | 0.1 |
| | | 14 | 0.6 | 99.3 | 0.1 |

Drug Product Formulation in liquid as well as in lyophilized forms were found stable at stress condition up to 14 days for lower and higher concentration (40 μg/mL and 240 μg/mL). Results are applicable for all in intermediary strengths.

### Example 3: Addition of a Chelator Stabilizes the Formulation

Subvisible particles in a formulation may consist of aggregated proteins and can directly impact the efficacy and immunogenicity of a drug product. Also, they often act as nucleation sites for further protein aggregation and/or lead to the development of larger particles by agglomeration (see, chapter 2.9.19. Particulate contamination: sub-visible particles has been published in Pharmeuropa 33.2).

To assess the impact of a chelator such as EDTA or DTPA on the stability of the formulation of Example 1, a sub-visible particle count analysis was performed at 37° C. using a HIAC 9703+ liquid particle counter system. The following formulations were tested: SON080F49a (without EDTA or DTPA), SON080F49b (with DTPA), and SON080F49c (with EDTA). The SON080F49a formulation comprises 2 mg/mL IL-6, 10 mM histidine buffer, 10 mg/mL glycine, 50 mg/mL trehalose, 0.2 mg/mL Polysorbate 20, water for injection, and has a pH 7.2.

| | Sample | | | | | | | | | | | |
|---|---|---|---|---|---|---|---|---|---|---|---|---|
| | SON080F49a | | | | | | | | | | | |
| Particle | (W/O EDTA or DTPA) | | | | SON080F49b (With DTPA) | | | | SON080F49c (With EDTA) | | | |
| size (μm) | Day 0 | Day 1 | Day 7 | Day 14 | Day 0 | Day 1 | Day 7 | Day 14 | Day 0 | Day 1 | Day 7 | Day 14 |
| 2 | 202 | 150 | 132 | 1553 | 198 | 225 | 300 | 272 | 113 | 322 | 215 | 425 |
| 5 | 68 | 32 | 45 | 355 | 57 | 73 | 110 | 65 | 22 | 67 | 53 | 90 |
| 10 | 32 | 10 | 8 | 68 | 30 | 18 | 47 | 30 | 20 | 23 | 17 | 50 |
| 25 | 0 | 0 | 2 | 3 | 2 | 2 | 3 | 2 | 0 | 3 | 0 | 0 |

The addition of a chelator to the SON080F49a formulation decreased large sub-visible particles of 2, 5, 10, and 25 μm at days 1, 7, and 14. Furthermore, the formulation comprising DTPA (SON080F49b) unexpectedly decreased sub-visible particle count as compared to the formulation with EDTA (SON080F49c).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, the group for reasons of convenience and/or patentability. When any such inclusion or deletion occurs, the specification is deemed to contain the group as modified, thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. An IL-6 formulation, comprising:
about 40 μg/mL to about 2 mg/mL IL-6;
about 5 to about 15 mM histidine;
about 5 to about 15 mg/mL glycine;
about 40 to about 60 mg/mL trehalose;
about 0.1 to about 0.3 mg/mL polysorbate 20; and
about 1 to about 1000 μM DTPA,
wherein the formulation has a pH of about 6.0 to about 8.0.

2. The IL-6 formulation of claim 1, wherein the IL-6 is human IL-6.

3. The IL-6 formulation of claim 1, wherein the formulation comprises 40 μg/mL IL-6, 45 μg/mL IL-6, 50 μg/mL IL-6, 55 μg/mL IL-6, 60 μg/mL IL-6, 65 μg/mL IL-6, 70 μg/mL IL-6, 75 μg/mL IL-6, 80 μg/mL IL-6, 85 μg/mL IL-6, 90 μg/mL IL-6, 95 μg/mL IL-6, 100 μg/mL IL-6, 105 μg/mL IL-6, 110 μg/mL IL-6, 115 μg/mL IL-6, 120 μg/mL IL-6, 125 μg/mL IL-6, 130 μg/mL IL-6, 135 μg/mL IL-6, 140 μg/mL IL-6, 145 μg/mL IL-6, 150 μg/mL IL-6, 155 μg/mL IL-6, 160 μg/mL IL-6, 165 μg/mL IL-6, 170 μg/mL IL-6, 175 μg/mL IL-6, 180 μg/mL IL-6, 185 μg/mL IL-6, 190 μg/mL IL-6, 195 μg/mL IL-6, 200 μg/mL IL-6, 205 μg/mL IL-6, 210 μg/mL IL-6, 215 μg/mL IL-6, 220 μg/mL IL-6, 225 μg/mL IL-6, 230 μg/mL IL-6, 235 μg/mL IL-6, or 240 μg/mL IL-6.

4. The IL-6 formulation of claim 3, wherein the formulation comprises 80 μg/mL IL-6.

5. The IL-6 formulation of claim 3, wherein the formulation comprises 120 μg/mL IL-6.

6. The IL-6 formulation of claim 3, wherein the formulation comprises 240 μg/mL IL-6.

7. The IL-6 formulation of claim 1, wherein the formulation comprises about 10 mM histidine.

8. The IL-6 formulation of claim 1, wherein the formulation comprises about 10 mg/mL glycine.

9. The IL-6 formulation of claim 1, wherein the formulation comprises about 50 mg/mL trehalose.

10. The IL-6 formulation of claim 1, wherein the formulation comprises about 0.2 mg/mL polysorbate 20.

11. The IL-6 formulation of claim 1, wherein the formulation comprises about 50 μM DTPA.

12. The IL-6 formulation of claim 1, wherein the formulation has a pH of about 7.0.

13. An IL-6 formulation, comprising:
40 to about 240 μg/mL human IL-6;
10 mM histidine;
10 mg/mL glycine;
50 mg/mL trehalose;
0.2 mg/mL polysorbate 20; and
50 μM DTPA,
wherein the formulation has a pH of about 7.0.

14. The IL-6 formulation of claim 13, wherein the formulation comprises 40 μg/mL IL-6, 45 μg/mL IL-6, 50 μg/mL IL-6, 55 μg/mL IL-6, 60 μg/mL IL-6, 65 μg/mL IL-6, 70 μg/mL IL-6, 75 μg/mL IL-6, 80 μg/mL IL-6, 85 μg/mL IL-6, 90 μg/mL IL-6, 95 μg/mL IL-6, 100 μg/mL IL-6, 105 μg/mL IL-6, 110 μg/mL IL-6, 115 μg/mL IL-6, 120 μg/mL IL-6, 125 μg/mL IL-6, 130 μg/mL IL-6, 135 μg/mL IL-6, 140 μg/mL IL-6, 145 μg/mL IL-6, 150 μg/mL IL-6, 155 μg/mL IL-6, 160 μg/mL IL-6, 165 μg/mL IL-6, 170 μg/mL IL-6, 175 μg/mL IL-6, 180 μg/mL IL-6, 185 μg/mL IL-6, 190 μg/mL IL-6, 195 μg/mL IL-6, 200 μg/mL IL-6, 205 μg/mL IL-6, 210 μg/mL IL-6, 215 μg/mL IL-6, 220 μg/mL IL-6, 225 μg/mL IL-6, 230 μg/mL IL-6, 235 μg/mL IL-6, or 240 μg/mL IL-6.

15. The IL-6 formulation of claim 14, wherein the formulation comprises 80 μg/mL IL-6.

16. The IL-6 formulation of claim 14, wherein the formulation comprises 120 μg/mL IL-6.

17. The IL-6 formulation of claim 14, wherein the formulation comprises 240 μg/mL IL-6.

18. The IL-6 formulation of claim 13, wherein the formulation is suitable for parenteral administration.

19. The IL-6 formulation of claim 13, wherein the formulation is suitable for intravenous administration.

20. The IL-6 formulation of claim 13, wherein the formulation is suitable for subcutaneous administration.

21. A method of treating a disease or disorder in a subject in need thereof, the method comprising administering the formulation of claim 1 to the subject.

22. The IL-6 formulation of claim 1, wherein the formulation has a post peak of less than about 3% after 14 days at −80° C. measured by RP-HPLC.

23. The IL-6 formulation of claim 1, wherein the formulation has a post peak of less than about 3% after 14 days at 2-8° C. measured by RP-HPLC.
